(19) United States
(12) Patent Application Publication
(10) Pub. No.: US 2003/0092027 A1
(43) Pub. Date: May 15, 2003

(54) Nuclear magnetic resonance method for identifying ligands to target compounds

(76) Inventors: Luciano Mueller, Princeton, NJ (US); Li-An Xu, Lawrenceville, NJ (US); Chin-Hua Frank Shen, Yardley, PA (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/159,351

(22) Filed: May 31, 2002

Related U.S. Application Data
(60) Provisional application No. 60/295,267, filed on Jun. 1, 2001.
(60) Provisional application No. 60/322,536, filed on Sep. 14, 2001.

(51) Int. Cl. C12Q 1/68; G01N 33/53; G06F 19/00; G01N 33/48; G01N 33/50

(52) U.S. Cl. 435/6; 435/7.1; 702/19; 702/20

(57) ABSTRACT

The invention provides methods of using two-dimensional nuclear magnetic resonance to identify compounds that bind to a target compound.

This application claims benefit to provisional application U.S. Serial No. 60/295,267 filed Jun. 1, 2001 and to provisional application U.S. Serial No. 60/322,536, filed Sep. 14, 2001. The entire teachings of the referenced applications are incorporated herein by reference.

### FIELD OF THE INVENTION

The present invention relates to methods for identifying ligands that bind to target molecules using two-dimensional nuclear magnetic resonance correlation spectral analysis.

### BACKGROUND OF THE INVENTION

Various technological advances have created an increased demand for methods to screen test compounds for interaction with a target compound. Combinatorial chemistry, for example, allows for the creation of large numbers of compounds for testing in drug discovery. Drug candidate compounds are now produced with increased efficiency and throughput. Moreover, the recent sequencing of the human genome has resulted in greater demands in the field of proteonomics—the study of the structure and function of proteins. Some estimates place the total number of human proteins in cells and tissues at about 12 million. Thus, there is an increased demand for methods for rapid, efficient, accurate and reliable methods of identifying compounds that bind to target molecules, especially proteins and nucleic acids.

Several approaches to screening a mixture of test compounds for interaction with target compounds have been described, including iterative rescreening of subsets of the mixture, recursive devolution, tagging, affinity mass spectroscopy, and affinity nuclear magnetic resonance (“NMR”). In particular, NMR spectroscopy has become a powerful technique for determining macromolecular structures of biological interest. NMR methods have been used to study the structure of proteins in solution, as well as the interaction of proteins and ligands. Various methods examine peak positions in proton:nitrogen nuclear magnetic resonance (“¹⁵N/¹H NMR”) correlation spectra of proteins. These peak positions are sensitive to perturbations induced by the binding of small organic molecules, such as substrates of drug molecules.

One approach, disclosed in U.S. Pat. No. 5,804,390, compares the ¹⁵N/¹H NMR correlation spectrum of a ¹⁵N-labeled protein with the ¹⁵N/¹H NMR correlation spectrum of a ¹⁵N-labeled protein that has been exposed to one or more compounds. In an ideal situation, differences in peak coordinates of the unexposed protein spectrum and the exposed protein spectrum indicate binding of at least one compound. However, this approach has several drawbacks. For example, this method is typically used to test up to ten compounds at a time, and thus fails to provide an efficient or high throughput screening method. In addition, when this method indicates that at least of the compounds in a mixture is active, each compound in the mixture must be individually tested for binding to the target protein. Furthermore, this method requires the generation of a reference NMR spectrum of the pure target compound in the absences of test compounds and relies on visual inspection for interpretation of the results.

Another approach attempts to identify non-significant peaks in NMR spectra (A. Ross et al., J. Biomol. NMR, 16, 139-146 (2000)). This approach requires identification and selection of cross peaks in proton:nitrogen correlation spectra. However, complications in peak selection arise when this approach is used with larger proteins, which give rise to spectra with dense clustering of cross peaks. Dense clustering of cross peaks also complicates the principal component analysis, because it may be difficult to properly trace chemical shift perturbations.

Due to the numerous problems with existing screening methods, there is a need for rapid, accurate, and reliable methods for identifying ligands that bind to target molecules. Specifically, there is a need for screening methods which have high throughput, do not require screening each compound in a mixture to identify active test compounds, do not require generation of a reference spectrum of the pure target compound in the absences of test compounds, and do not require manual selection of cross peaks in two dimensional NMR spectra.

### SUMMARY OF THE INVENTION

The invention features a method of identifying test compounds that interact with a target compound. In particular, the present invention uses two-dimensional NMR to identify compounds that interact with a target compound. In one aspect, the invention utilizes ¹⁵N/¹H NMR correlation spectra or ¹³C/¹H NMR correlation spectra of an appropriately labeled target compound and a plurality of test compounds to identify non-significant peaks, or reference spectra, in the spectra. Non-significant peaks are excised from the spectra, revealing spectra that indicate interactions between the target compound and one or more test compounds. The method is used with mixtures or libraries of test compounds to identify one or more test compounds that interact with a target compound. Such active test compounds may be identified without subsequently testing each compound individually for binding to the target protein. In preferred embodiments, the target compound is a protein. In another preferred embodiment, non-significant peaks are identified by statistical analysis. The invention permits automated identification of those spectra where precipitation of target compounds due to interaction with a test compound produced significant losses in overall spectral signal intensities.

In another aspect, the invention features a method of identifying test compounds that interact with a target compound using an expectation maximization (EM) algorithm. In this aspect, the method of the invention includes generating two-dimensional spectra of a plurality of test compounds in the presence of a target compound, and analyzing the data with an expectation maximization algorithm.

In preferred embodiments, the invention features a method for pre-processing spectral data to eliminate singleton peaks. In yet other preferred embodiments of the invention, spectral data is normalized.

### BRIEF DESCRIPTION OF THE DRAWINGS

FIG. 1 depicts the superposition of 78 ¹⁵N/¹H NMR correlation spectra recorded in mixtures of uniformly ¹⁵N-labeled peptidyl deformylase and test compounds.

FIG. 2 depicts the superposition of the 39 raw reference ¹⁵N/¹H NMR correlation spectra (single contour per spectrum) recorded in mixtures of ¹⁵N-labeled peptidyl deformylase and compounds which are known not to bind peptidyl deformylase.

FIG. 3 depicts the superposition of the same 39 spectra which are depicted in FIG. 2. Peaks which exhibit considerable variations are highlighted by rectangular boxes.

FIG. 4 depicts the superposition of the refined set of reference spectra using the median of the median method.

FIG. 5 depicts superposition of the 19 refined reference spectra each plotted with 8 contours with spectrum 14, plotted with a single contour.

FIG. 6 depicts the superposition of 19 refined reference spectra, each plotted with 8 contours. The contours of the last spectrum are in white, and the five regions which are boxed in FIG. 3 are nulled (purged spectrum).

FIG. 7 depicts the superposition of the 19 refined reference spectra plus spectrum 14. The reference spectra are plotted with 8 contours and spectrum 14 is plotted with 1 contour.

FIG. 8 depicts the pair-wise T-score between a reference spectrum and all other spectra in the ensemble of ¹⁵N/¹H NMR correlation spectra.

FIG. 9A depicts the difference between two spectra where there is no peak shift.

FIG. 9B depicts the difference between two spectra where there is peak shift.

FIG. 10 depicts the difference between raw (+) and filtered data (∘), after median filtering.

FIG. 11A depicts the Q-Q plot for normal distribution.

FIG. 11B depicts the Q-Q plot for exponential distribution.

FIG. 12 depicts the boxplot of the Tₖ for each of the 78 spectra.

FIG. 13 depicts the normalization procedure.

### DETAILED DESCRIPTION

The invention provides a method for the rapid and efficient identification of molecules that interact with target molecules. The method is thus useful for identifying ligands to a target compound, such as a protein, and may be used for discovering new drug leads. The method of the invention is used to screen mixtures or libraries of test compounds to identify one or more test compounds that interact with a target compound, without the need to test each individual compound subsequently for binding to the target protein. The method eliminates the need for recording an NMR spectrum of the target compound in the absence of test compounds. Instead, the method utilizes a reference spectrum which arises from the statistical analysis of an ensemble of spectra which are recorded in mixtures of target compound and test compounds. In addition, the method yields unambiguous identification of target and test compound interaction, which produce distinct perturbations in a two dimensional spectrum, such as ¹⁵N/¹H NMR correlation spectra or ¹³C/¹H NMR correlation spectra. The method thus reduces the need for visual inspection on a subset of spectra where the statistical protocol gives rise to an ambiguous result. Furthermore, the method of the invention does not require the explicit selection of cross peaks in NMR spectra.

Before further describing the present invention, the following definitions are provided for terms used in this specification:

Definitions

“Non-significant peak” refers to a peak in a number of two-dimensional correlation spectra which varies its position along relatively straight lines in the spectral plane in a sizeable fraction of the spectra.

“Reference spectrum” refers to a two-dimensional correlation spectrum of a target compound with no ligand interactions, i.e., a target compound in the presence of compounds which do not interact with the target. Thus, as used herein, “a non-significant peak” is a reference spectrum identified by visual inspection of a plurality of two-dimensional correlation spectra.

“Refined reference spectra” refers to reference spectra identified or selected by a statistical method.

“Resulting spectrum” refers to a two-dimensional correlation spectrum of a target compound and test compounds which has had reference spectra or refined reference spectra excised.

“Target compound” refers to any molecule typically enriched with nitrogen-15 or carbon-13 isotopes, as appropriate, that may be analyzed by two-dimensional NMR, i.e., a molecule that generates a two-dimensional NMR correlation spectrum. Preferred target compounds include proteins, peptides, and nucleic acids.

“Test compound” refers to any molecule exposed to a target compound to determine whether or not the target compound interacts with that molecule.

“Two dimensional NMR” or “2D NMR” refers to any nuclear magnetic resonance spectra presented in two dimensions, such as correlation spectra. Preferred 2D NMR methods are ¹⁵N/¹H NMR and ¹³C/¹H NMR.

The present invention uses two-dimensional NMR to identify compounds that interact with a target compound. The present invention takes advantage of the premise that, within a chemically diverse mixture of test compounds, only a relatively small subset possesses detectable binding affinities to a target compound. Thus, in a plurality of two-dimensional NMR spectra of a mixture of target and test compound, peaks which vary their coordinates are not likely associated with specific interaction between the target compound and the test compound. Such “non-significant peaks” are readily identifiable because they tend to vary their coordinates along straight lines in a spectral plane. By analyzing the chemical shift changes in a plurality of spectra, non-specific interactions may be eliminated from consideration, thereby focusing the analysis on spectra which indicate specific interactions between target and test compound.

In one aspect, the invention features a method of identifying compounds that bind to a target compound. The method includes the steps of generating a plurality of two-dimensional NMR correlation spectra of a target compound that has been exposed to a plurality of test compounds, identifying a reference spectrum in the plurality of two-dimensional NMR correlation spectra, and excising the reference spectrum from the plurality of two-dimensional NMR correlation spectra to create a resulting spectrum, wherein the resulting spectrum identifies one or more of the test compounds that interact with the target compound. In one preferred embodiment, the plurality of two-dimensional NMR correlation spectra is a plurality of ¹⁵N/¹H NMR correlation spectra. Preferred target compounds include proteins, peptides, and nucleic acids.

In one preferred embodiment of the invention, the step of identifying a reference spectrum includes the step of analyzing the plurality of two-dimensional NMR correlation spectra using a statistical method. Preferably, the statistical method includes the step of performing a cluster analysis. Most preferably, the cluster analysis includes the step of performing a T-score analysis. A T-score analysis used in the method of the invention preferably uses either a second quartile or third quartile to determine a cutoff point for the T-score analysis.

In another preferred embodiment of the invention, the step of excising the reference spectrum includes analyzing the plurality of two-dimensional NMR correlation spectra and the reference spectrum using an EM algorithm. In some embodiments, the method of the invention includes the step of normalizing the plurality of two-dimensional NMR correlation spectra. In other embodiments, the method of the invention includes the step of eliminating singleton peaks in the plurality of two-dimensional NMR correlation spectra.

In a preferred embodiment, the plurality of spectra is subjected to statistical analysis to identify reference spectra. Most preferably, the statistical method used to identify reference spectra is a quartile adaptive method, as described herein. For example, when there is little information about the number of peak shifts in the plurality of spectra, the second quartile of the medians or “median of the medians” method may be used to identify reference spectra. Where it is known that there are fewer peak shifts in the plurality of spectra, the data set may be analyzed using third quartile of the medians. Reference spectra identified by statistical analysis are referred to herein as “refined reference spectra.”

A preferred two-dimensional NMR method is two-dimensional ¹⁵N/¹H NMR. Any ¹⁵N-labeled target compound may be used to generate a ¹⁵N/¹H NMR correlation spectrum. Suitable ¹⁵N-labeled target compounds may be prepared by known methods utilizing over-expression in isotopically enriched growth media or chemical synthesis using isotopically enriched chemical building blocks, or obtained from commercial sources. Means for generating two dimensional NMR spectra, such as ¹⁵N/¹H NMR correlation spectra, are well known in the art. Mixtures of target compound and test compounds are formed by combining the target compound, either simultaneously or sequentially, with the test compounds under suitable conditions. Typically, stock solutions of tests compounds are prepared at relatively high concentration (typically in excess of 30 mM) in versatile organic solvents such as dimethyl sulfoxide (DMSO). Aliquots of stock solution are added to a buffered protein solution. Protocols for preparing buffered solutions of target compounds, the preparation of stock solutions of test compounds, and the generation mixtures of suitable mixtures of solutions containing target compound and tests are well known in the art.

Confirmation of the interaction between target and test compound may be performed by methods well known in the art, such as X-ray crystallography. The three dimensional structure of the complexed target and test compound may also be determined by known methods.

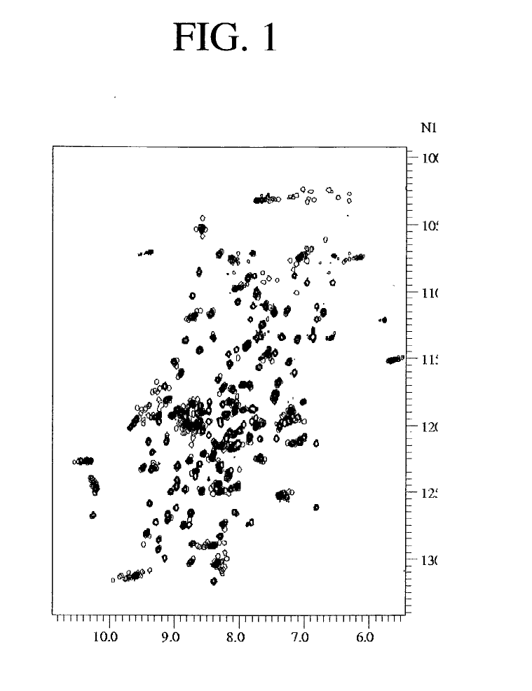

FIG. 1 shows the superposition of 78 ¹⁵N/¹H NMR correlation spectra, as described in Example 1. The spectra are generated from mixtures of ¹⁵N-labeled peptidyl deformylase and 78 test compounds. Each peak represents the cross correlation of references between proton and nitrogen pairs.

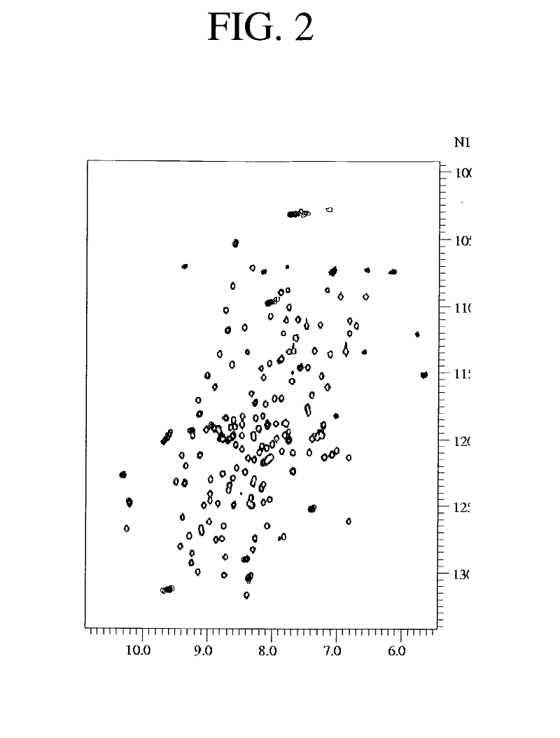

The 39 spectra of compounds known not to interact with peptidyl deformylase are shown in FIG. 2. These are the spectra for sample numbers 3, 4, 9, 12, 13, 16, 17, 18, 21, 23, 25, 26, 30, 31, 32, 35, 38, 39, 40, 41, 42, 43, 44, 49, 50, 51, 52, 53, 54, 55, 58, 59, 61, 62, 65, 66, 67, 75, and 78. A single contour is shown for each spectrum.

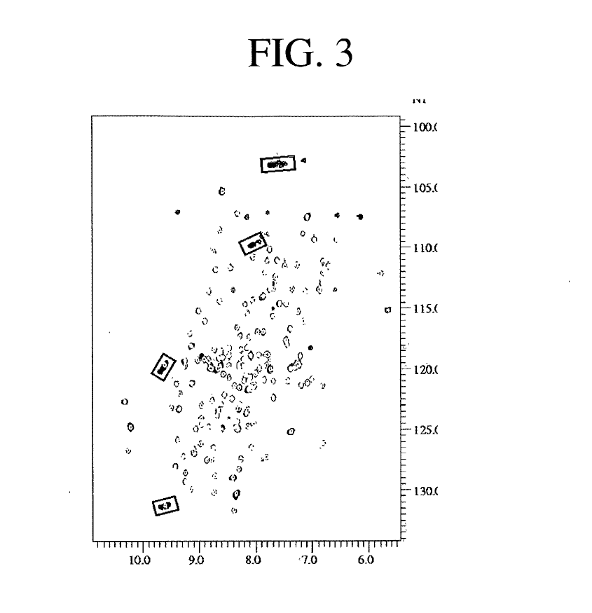

FIG. 3. shows the superposition of the 39 spectra depicted in FIG. 2. Each spectrum is plotted with a set of geometrically spaced contours using a uniform spacing of 1.3 between contour levels. The last spectrum (sample #78) is plotted with white contours. In FIG. 3, overlapping white contours blank out the black contours of preceding spectra. Faint cross peaks thus indicate good overlap among all spectra, while black contours are indicative of variability of cross peak coordinates. Peaks which exhibit considerable variation are outlined by rectangular boxes.

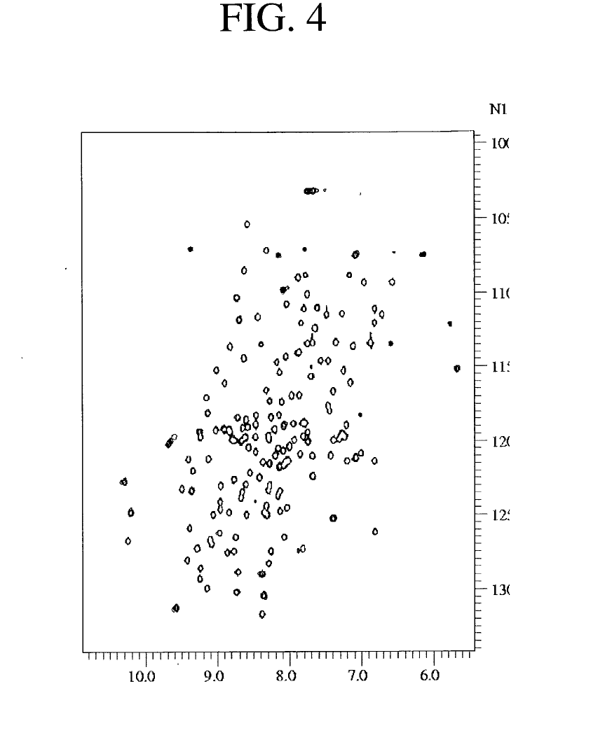

FIG. 4 shows the superposition of the refined reference spectra using the second quartile or median of the median method. The 19 spectra identified by this method correspond to sample numbers 4, 9, 16, 18, 23, 31, 32, 35, 40, 41, 44, 50, 51, 52, 58, 59, 61, 62, and 66. This method identified 19 of the 39 known reference spectra, or approximately 50% of the reference spectra. The misclassification rate is thus about 8%. Using the third quartile of the median, 29 of the 39 (about 75%) known reference spectra were identified, with a resulting misclassification rate of 0%.

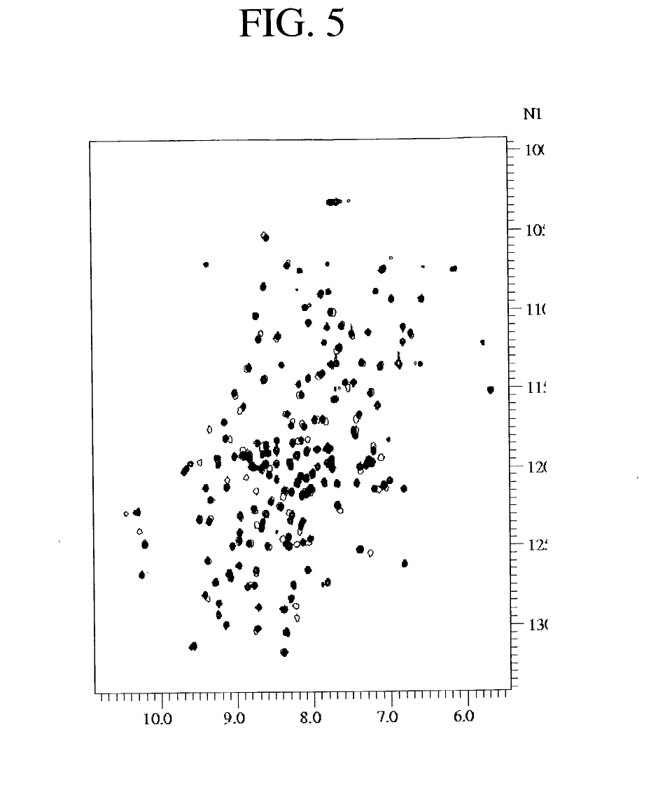

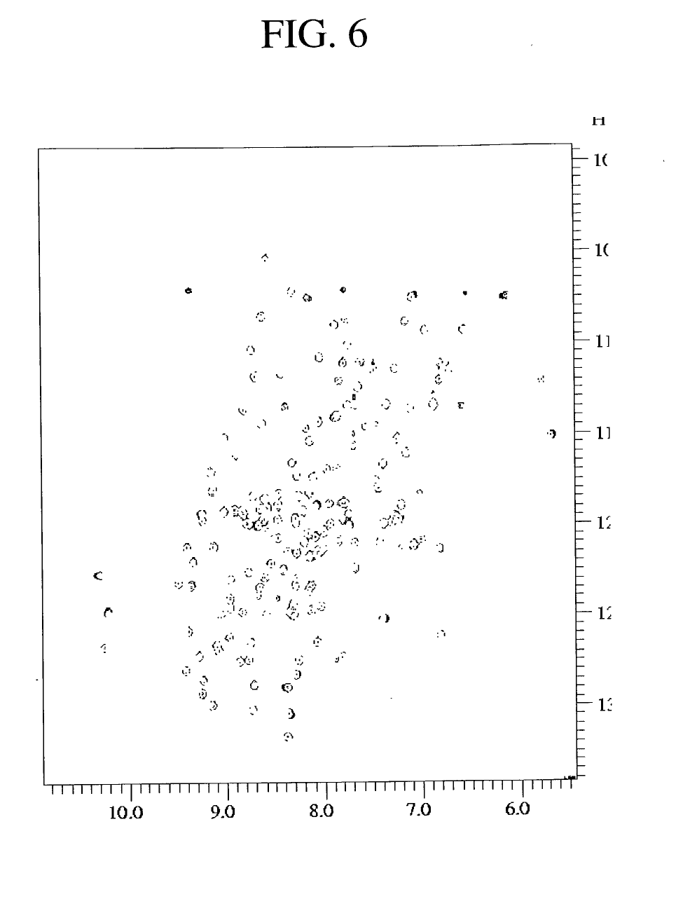

FIG. 5 shows the superposition of the refined 19 reference spectra, each plotted with 8 contours, and spectrum 14, which represents a test compound that interacts with the target compound, plotted with a single contour. FIG. 6 shows the superposition of 19 refined reference spectra, each plotted with 8 contours. The contours of the last spectrum are in white. The five regions which are boxed in FIG. 3 are nulled (purged spectrum).

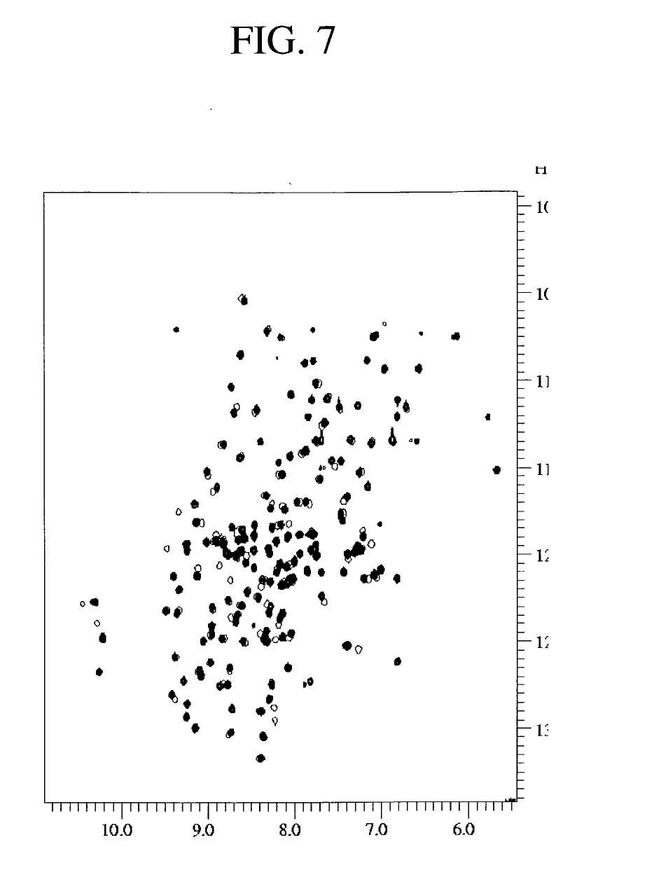

FIG. 7 shows the superposition of 19 refined reference spectra and spectrum 14. The refined reference spectra are plotted with 8 contours and spectrum 14, which represents a test compound that interacts with the target compound, is plotted with a single contour.

Statistical Analysis

In a preferred embodiment of the present invention, statistical analysis is employed to detect whether there are peak shifts in the resulting spectrum, as compared to a reference spectrum or spectra. In preferred embodiments, the invention uses statistical methods to identify reference spectra. If desired, data may be pre-processed as described herein.

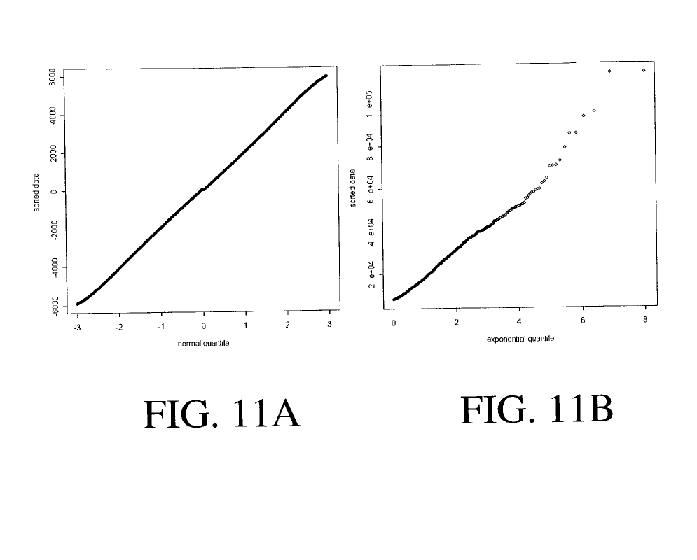

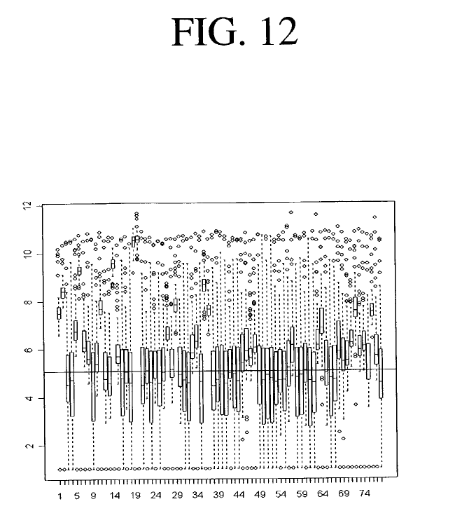

The statistical model used assumes that the difference between the resulting spectrum and the reference spectrum or spectra can be modeled as a mixture of Gaussian and double exponential distributions. The Gaussian distribution represents random noise in the difference resulting from no peak shifts, while the double exponential component delineates differences from those peaks shifted in the resulting spectrum. FIGS. 11A and 11B suggest that this assumption holds.

Specifically, let Xᵢ be the i-th difference of two spectra. It has the following density function

$$pf ( x \vert α ) + ( 1 - p ) φ ( \frac{x}{σ} ) , \qquad ( 1 )$$

where f(x|α) is the probability density function for a double exponential distribution, φ(.) is the probability density function for a standardized Gaussian distribution, p is the proportion that x belongs to the double exponential distribution, and α and σ are other parameters of interest. Using this model, the likelihood function of X is as follows:

$${\prod }_{i } ( p \frac{α}{4} exp \{ - α \vert {x}_{i} \vert \} + ( 1 - p ) \frac{1}{\sqrt{2 π} σ} exp \{ - \frac{{x}_{i}^{2}}{2 {σ}^{2}} \} ) \qquad ( 2 )$$

An iterative EM algorithm (see Dempster et al., 1977) is used to obtain the maximum likelihood estimates (MLE) of the parameters p, α and σ for the mixture model. Here E stands for expectation and M stands for maximization. To implement the EM algorithm, an indicator variable zᵢ is introduced, where zᵢ=1 if the i-th observation is from the double exponential distribution, and zᵢ=0 if the i-th observation is from the normal distribution. Thus, the complete log-likelihood function of X and z is:

$$l = - α {\sum }_{i {z}_{i} = 1} \vert {x}_{i} \vert + ( {\sum }_{i} {z}_{i} ) log ( α ) - ( n - {\sum }_{i} {z}_{i} ) log ( σ ) - {\sum }_{i {z}_{i} = 0 } \frac{{x}_{i}^{2}}{2 {σ}^{2}} + ( {\sum }_{i} {z}_{i} ) log ( p ) + ( n - {\sum }_{i} {z}_{i} ) log ( 1 - p ) . \qquad ( 3 )$$

Parameters p, α and σ will be estimated iteratively from equation (3).

Data Pre-Processing

Data may be pre-processed by normalization and/or singleton elimination techniques.

Normalization

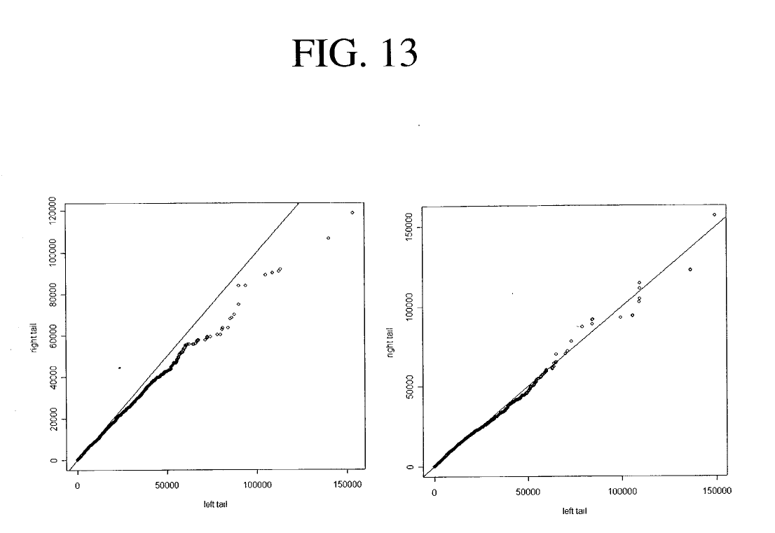

Raw data may be normalized. The difference of the magnitude due to measurement errors can raise a serious problem because the present method uses the difference of the two spectra to detect the peak shifts. The magnitude of one spectrum may be shrunk more severely than that of another spectrum in the experiment. Furthermore, the shrinking effects on peaks and noise may be quite different. To make spectra more comparable, the data can be normalized using the following normalization procedure. First, a robust estimate of the variance of the noise for each spectrum is obtained using the MAD estimator described in Modern Applied Statistics with S-PLUS, W. N. Venables, B. D. Ripley, Springer, 1997. Second, in each spectrum the pixels whose magnitude are 6 times greater than its MAD are identified. Median magnitude of these pixels is calculated for each spectrum. These pixels identified in each spectrum are then scaled by dividing its own median and multiplying the mean of all the medians. For the pixels whose magnitude is 6 times less than the MAD of that spectrum, the same procedure is used to scale them. An example of the improvement of applying the normalization procedure is showed in the FIG. 13. In this example two spectra are normalized. If these two spectra have same magnitude (no shrinking effects on the spectra), in the distribution of the difference spectrum on the two tails should be symmetric. Thus, the scatter plot of the two tails should fall on the 45-degree line. The left panel of FIG. 13 shows the two tails of the difference spectrum somehow are not symmetric before the normalization. The right panel of FIG. 13 is the same plot after the normalization.

Singleton Elimination

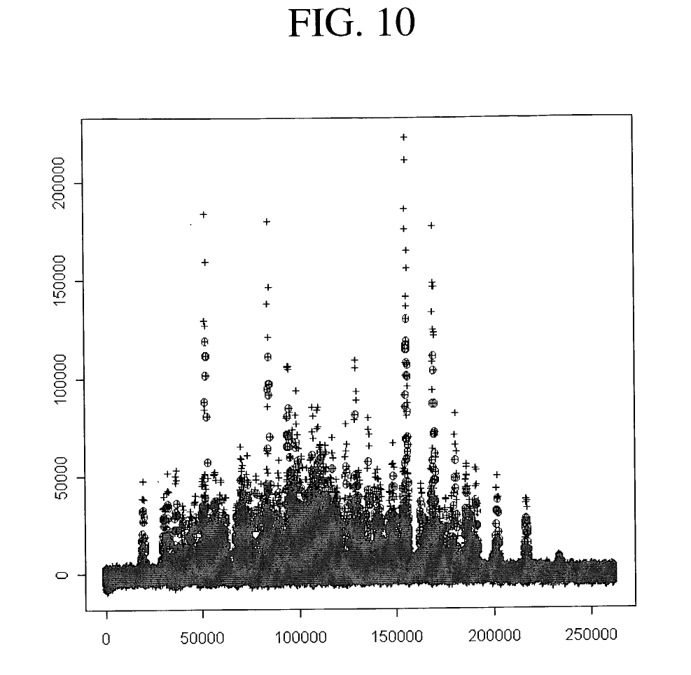

One potential problem in the raw spectral data is that it may contain isolated pixels (“singleton peaks”) with very high magnitude, while adjacent pixels have very low magnitude. These singleton peaks are eliminated by a median filter method. Namely, at each pixel, a window is first selected and the median of the data within that window is calculated. Then the value of that pixel is then assigned either (1) the median; or (2) the average of the window without pixel values that are 3 times greater than the median. FIG. 10 shows the difference between the raw data (crosses) and the data after median filtering (circles).

Estimating Model Parameters p, α, and σ.

The EM algorithm is used to estimate parameters p, α, and σ. Other estimation methods known in the art can be applied for the same purpose.

Initial Estimates of p, α, and σ

The top 10% of the pre-processed data is truncated and the standard deviation of the remainders is used as the initial estimate for σ. The truncating percentage may range from 1% to 90%, depending on the data. In most instances, the percent truncation will range between 5% and 40%, but most preferably is about 10%. The initial estimates for p and α are 0.5 and 3 times the value of σ, respectively.

The E-Step

The E-step of the EM algorithm obtains the probability of the peak shifts (i.e., locations followed the double exponential distribution) from the following equation

$$P ( {z}_{i} = 1 \vert α , σ , p ) = \frac{pf ( x ; α )}{pf ( x ; α ) + ( 1 - p ) φ ( x / σ )} \qquad ( 4 )$$

with parameters α, p and σ estimated as described above for the first pass, and subsequently from the M-step described below.

The M-Step

The M-step of the EM algorithm maximizes the complete likelihood function in equation (3). It yields the following updating equations:

$$\hat{p} = \frac{\sum P ( {z}_{i} = 1 )}{n} , \hat{α} = \frac{\sum P ( {z}_{i} = 1 )}{\sum {x}_{i} P ( {z}_{i} = 1 )} , {\hat{σ}}^{2} = \frac{\sum {x}_{i}^{2} P ( {z}_{i} = 0 )}{\sum P ( {z}_{i} = 0 )} \qquad ( 5 )$$

The parameters α, p and σ are repeatedly updated until a preset convergence criterion is satisfied.

Inference Using T-Score

To test the following hypothesis:

H₀: No peak shifts in the resulting spectrum, versus

H₁: there is at least one peak shift in the resulting spectrum, let

$${T}_{k} = \frac{\begin{matrix}proportion of observations \\ greater than k \hat{σ} or less than - k \hat{σ}\end{matrix}}{2 ( 1 - Φ ( k ) )} \qquad ( 6 )$$

where k is usually set to be 3, and Φ(.) is the cumulative density function of standard normal distribution. The expected value of Tₖ is 1 under the null hypothesis H₀. A large T-score therefore suggests a possibility of peak shifts in the treatment spectrum. A T-score greater than a preset cutoff value would reject H₀ and conclude there are peak shifts in that spectrum. The appropriate cutoff value to use is: \overline{T}_{k,r}+3\hat{σ}_{k,r}, where \overline{T}_{k,r} and \hat{σ}_{k,r} are the mean and standard deviation estimated from the empirical distribution of Tₖ calculated for the reference spectrum set as described below.

Reference Spectrum Identification

In a preferred embodiment, a statistical method is used to identify a set of spectra which have very little peak shifts and therefore can be served as references. Assuming that n spectra are observed, each spectrum is treated as a hypothetical reference and the n-1 T-scores are calculated using equation (6). The median of the n-1 T-scores of each spectrum is determined and called: Mᵢ,i=1, . . . , n. A cutoff point is then based on one of the quartiles for n medians obtained. For example, choosing the second quartile yields the cutoff point as the median of Mᵢ's (“median of the median”). Spectrum i is included as a reference spectrum if Mᵢ is less than the cutoff point.

### EXAMPLES

### Example 1

### Convergence of the EM Algorithm

This example illustrates how the EM algorithm works when one treatment spectrum is compared to the reference spectrum. As shown in Table 1, the initial estimates for p, α, and σ are 0.3, 2127.707, and 0.000157, respectively. The convergence after 60 iterations with a convergent criterion of that the difference of the two consecutive estimates are less than 0.001. The same convergence was observed by using different initial values for p, α, and σ which suggested the robustness of the EM algorithm.

TABLE 1
The convergence of the estimate of ρ, α, and σ
in the EM algorithm.

| Iterations | ρ | σ | α |
|---|---|---|---|
| 1 | 0.3 | 2127.707 | 0.000157 |
| 2 | 0.137813 | 1874.852 | 0.000493 |
| 3 | 0.127749 | 1879.719 | 0.000476 |
| 4 | 0.118431 | 1886.818 | 0.000456 |
| 5 | 0.109588 | 1894.367 | 0.000438 |
| 6 | 0.101246 | 1902.107 | 0.000419 |
| 7 | 0.093428 | 1909.915 | 0.0004 |
| 8 | 0.086152 | 1917.707 | 0.000382 |
| 9 | 0.079433 | 1925.414 | 0.000364 |
| 10 | 0.073278 | 1932.966 | 0.000347 |
| 20 | 0.039693 | 1987.197 | 0.000236 |
| 30 | 0.033626 | 2001.145 | 0.000211 |
| 40 | 0.032903 | 2002.964 | 0.000208 |
| 50 | 0.032823 | 2003.166 | 0.000208 |
| 60 | 0.032814 | 2003.188 | 0.000208 |

### Example 2

### Peak Shift Detection

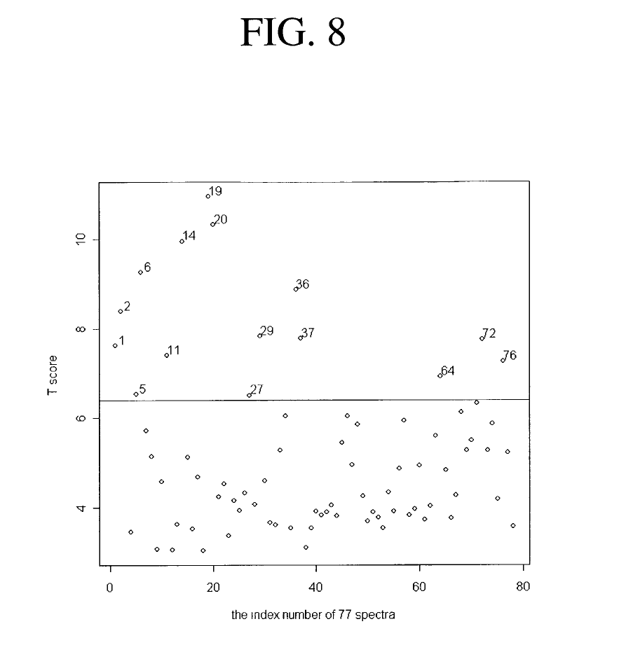

Seventy-eight spectra were included in this example. The spectrum of sample no. 3 is used as a reference spectrum: that is, the algorithm was applied and the T-scores calculated for all spectra with respect to the spectrum of sample no. 3. FIG. 8 depicts the results. Selecting 6.4 as the cutoff point for Tₖ, the spectra of sample nos. 1, 2, 5, 6, 11, 14, 19, 20, 27, 29, 36, 37, 64, 72, and 76 were identified to have peak shifts. This list includes all spectra having “true hits,” i.e., test compounds which were independently identified as interacting with the target compound, with the exception of spectrum 74. Upon careful inspection, it was concluded that spectrum 74 as a “hit” in the pre-processed data format. This list also contains two extra spectra, numbers 1 and 72, which show weak binding to the target compound, by the scientist's classification. Spectrum 20 is also of note in that it contains virtually no data because the test compound precipitated most of the protein, resulting in elimination of all cross peaks.

### Example 3

### Difference Between Two Spectra

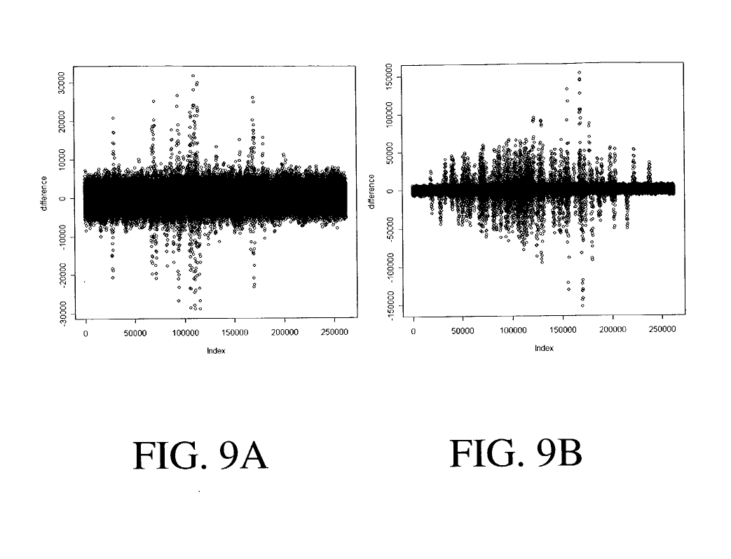

The difference between two spectra, as compared to spectrum 3, is demonstrated in FIGS. 9A and 9B. In particular, FIG. 9A shows no peak shifts in spectrum 9, whereas FIG. 5B shows peak shifts in spectrum 19. This is consistent with the results of the T-score analysis, as shown in FIG. 8.

### Example 4

### Refined Reference Spectra Determination

The following 39 spectra are identified as refined reference spectra after applying the method described above to the 78 spectra used in Example 2: 3, 4, 9, 12, 13, 16, 17, 18, 21, 23, 25, 26, 30, 31, 32, 35, 38, 39, 40, 41, 42, 43, 44, 49, 50, 51, 52, 53, 54, 55, 58, 59, 61, 62, 65, 66, 67, 75, and 78. FIG. 8 demonstrates the distribution of Tₖ for all spectra using a boxplot. The horizontal line in the graph is the median of the median, which is 5.076. The spectra whose median of Tₖ are under the line are selected as refined reference spectra.

### Example 5

### Misclassification Rate for the Refined Reference Spectra

In this example, the refined reference spectra identified in Example 4 are used as if they were the raw data set to determine whether there were peak shifts in these spectra. Using the “median of the median” method described above, 19 out 39 spectra were identified as reference spectra: 4, 9, 16, 18, 23, 31, 32, 35, 40, 41, 44, 50, 51, 52, 58, 59, 61, 62, and 66. Using the procedure described above, the cutoff point for Tₖ was 4.24. Only three spectra were determined to have peak shifts: 26, 42, and 75. The error rate was thus 3/39, or 7.7%. Where, as here, there is greater confidence that most of the spectra identified in Example 4 are reference spectra, the cutoff may be relaxed from the median (i.e., second quartile) to the 75th percentile (i.e., third quartile) to identify the refined reference spectra for this data set. Using the third quartile, 29 out of 39 reference spectra were identified and no peak shifts were identified. Hence the error rate is 0% in this case.

### Example 7

### The Programming Code for the Algorithm

In a preferred embodiment, the method of the invention is performed using a computer, with the algorithm written in the appropriate code. The programming code below is written in Splus. However, the algorithm may be written in any other software programming language, such as C, C++, Java, Visual Basic, Perl, SAS, SPSS, R, Fortran, MatLab, Maple, Mathematica, etc., by those skilled in the art.

The following is the Splus code for the algorithm:

# the function to do the median filtering
medianSmooth<-function (x, size=512 , ks=3)
{
img<-matrix(x, size, size)
imgnew<-matrix(0, size, size)
for (i in 2: (size-1))
for (j in 2: (size-1))

imgnew [i, j] <-median (img [ (i−1) : (i+1) , (j−1) : (j+1) ] )

for(j in 2: (size-1)) {

imgnew [1, j] <-median (img [1:2, (j−1) : (j+1) ] )
imgnew [size, j] <-median (img [(size-1) : size, (j−1) : (i+1) ] )

}
for(i in 2: (size-1)) {

imgnew [i, 1] <-median (img [ (i−1) : (i+1), 1:2] )
imgnew [i, size] <-median (img [ (i−1) : (i+1) , (size-1) :size] )

}
imgnew [1, 1] <-
median (c (img [1, 1], img [1, 2], img [2, 1], img [2, 2] ) )
imgnew [1, size] <-median (c (img [1, size-
1], img [1, size], img [2, size-1], img [2, size]
imgnew [size, 1] <-median (c (img [size-1, 1], img [size-
1, 2], img [size, 1], img [size, 2 ] )
imgnew [size, size] <-median (c (img [size-1, size-1], img [size-
1, size], img [size, size-1], img [size, size] ) )
as.vector (imgnew)
}
# the function to calculate the T-score for one
difference of the
# treatment spectrum and the reference
# spectrum using the EM algorithm
score.em<-function (diff, k=3)
{
# initial values
sig_sqrt (var (diff) )
tmp_diff [abs (diff) <3*sig]
s_sqrt (var (tmp) )
alpha_1/s/3
p_.5
for(i in 1:100) {
# E-step
prop_p*alpha*exp (-alpha*abs (diff) /2) /4
prop_prop/ (prop+ (1-p) *exp(-
diff*diff/2/s/s) /s/sqrt(2*3.14159) )
# M-step
p_sum(prop) /512/512
s_sqrt(sum(diff*diff* (1-prop)) /sum(1-prop) )
alpha_1/ (sum(abs (diff) * (prop) ) /2/sum (prop) )
}
score_length (diff [abs (diff) >3*s]) /512/512
score_score/ (1-pnorm(k) ) /2
res_list (score=score, p=p, s=s, alpha=alpha)
res
}
# calculate the T-scores for a set of treatment spectrum
by comparing
# the reference spectrum
nmrs.em_function (basenumber, k=3, start=1, end=78)
{

len_end-start+1
values_vector(“list”, len)
j_0
data.base_scan (paste (“spectrum”,basenumber, “.txt”,

collapse=“”, sep=“”) )

if(basenumber>end || basenumber<start) {
for(i in (start:end) )
{
j_j+1
data_scan (paste (“spectrum”, i, “.txt”,

collapse=“”, sep=“”) )

diff_medianSmooth(data) -medianSmoooth (data.base)
cat (i, “\n”)
values [ [j] ]_score.em(diff, k)
}
}
else{
for(i in (start:end) [-(basenumber-start+1) ] )
{
j_j+1
data_scan (paste (“spectrum”, i, “.txt”,

collapse=“”, sep=“”))

diff_medianSmooth (data) -medianSmooth (data.base)
cat (i, “\n”)
values [ [j] ]_score.em(diff, k)
}
}

values
}

Other Embodiments

From the foregoing description, it will be apparent that variations and modifications may be made to the invention described herein to adopt it to various usages and conditions.

Incorporation By Reference

All publications, patents, and patent applications mentioned in this specification are herein incorporated by reference to the same extent as if each publication, patent, or patent application was specifically and individually indicated to be incorporated by reference.

What is claimed is:

1. A method of identifying compounds that bind to a target compound, comprising:
a) generating a plurality of two-dimensional NMR correlation spectra of a target compound that has been exposed to a plurality of test compounds;
b) identifying a reference spectrum in the plurality of two-dimensional NMR correlation spectra; and
c) excising the reference spectrum from the plurality of two-dimensional NMR correlation spectra to create a resulting spectrum, wherein the resulting spectrum identifies one or more of the test compounds that interact with the target compound.

2. The method of claim 1, wherein the plurality of two-dimensional NMR correlation spectra is a plurality of ¹⁵N/¹H NMR correlation spectra.

3. The method of claim 1, wherein the target compound is selected from a protein, a peptide, and a nucleic acid.

4. The method of claim 1, wherein the step of identifying a reference spectrum comprises the step of analyzing the plurality of two-dimensional NMR correlation spectra using a statistical method.

5. The method of claim 4, wherein the statistical method comprises the step of performing a cluster analysis.

6. The method of claim 5, wherein the cluster analysis comprises the step of performing a T-score analysis.

7. The method of claim 6, wherein the T-score analysis includes using a second quartile of the medians to determine a cutoff point.

8. The method of claim 6, wherein the T-score analysis includes using a third quartile of the medians to determine a cutoff point.

9. The method of claim 1, wherein the step of excising the reference spectrum comprises analyzing the plurality of two-dimensional NMR correlation spectra and the reference spectrum using an EM algorithm.

10. The method of claim 1, further comprising the step of normalizing the plurality of two-dimensional NMR correlation spectra.

11. The method of claim 1, further comprising the step of eliminating singleton peaks in the plurality of two-dimensional NMR correlation spectra.
